(19) United States
(12) Patent Application Publication
(10) Pub. No.: US 2021/0282657 A1
(43) Pub. Date: Sep. 16, 2021

(54) METHOD FOR DYNAMICALLY SWITCHING BLOOD PRESSURE MEASUREMENT MODEL

(71) Applicants: Inventec (Pudong) Technology Corporation, Shanghai (CN); INVENTEC CORPORATION, Taipei (TW)

(72) Inventors: Trista Pei-Chun CHEN, Taipei (TW); Jonathan Hans Soeseno, Taipei (TW); Wei-Chao Chen, Taipei (TW)

(21) Appl. No.: 15/931,906

(22) Filed: May 14, 2020

(30) Foreign Application Priority Data
Mar. 15, 2020 (CN) 202010178885.X

(51) Int. Cl. A61B 5/022; A61B 5/024; A61B 5/0402; A61B 5/00

(52) U.S. Cl. CPC A61B 5/02225; A61B 5/02416; A61B 5/0402; A61B 2562/0219; A61B 5/7264; A61B 2562/085; A61B 5/6802

(57) ABSTRACT

A method for dynamically switching blood pressure measurement model, adapted to a wearable blood pressure measurement device with a biosignal sensing assembly and a processor, wherein said biosignal sensing assembly comprises two exposed electrodes, comprises: obtaining potential difference by said two exposed electrodes; determining whether potential difference is smaller than potential threshold by processor; obtaining first biosignal of specified user by biosignal sensing assembly when potential difference is smaller than the potential threshold; calculating first blood pressure value by processor according to at least first biosignal and first blood pressure model and outputting first blood pressure value; obtaining second biosignal of specified user by biosignal sensing assembly when potential difference is not smaller than the potential threshold, wherein types of first and second biosignal are different; and calculating second blood pressure value by processor according to second biosignal and second blood pressure model and outputting second blood pressure value.

### CROSS-REFERENCE TO RELATED APPLICATIONS

This non-provisional application claims priority under 35 U.S.C. § 119(a) on Patent Application No(s). 202010178885.X filed in China on Mar. 15, 2020, the entire contents of which are hereby incorporated by reference.

### BACKGROUND

### 1. Technical Field

This disclosure relates to a method for switching blood pressure measurement model, and more particularly to a method for dynamically switching blood pressure measurement model.

### 2. Related Art

Cardiovascular related diseases have been proven to be highly related to heart rate and blood pressure. Uncontrolled high blood pressure (BP) can lead to heart attack, stroke, heart failure, and other serious life threats. Therefore, accurate measurement of blood pressure is necessary to prevent unwanted events. According to the validation protocol followed by American National Standards Institute (ANSI), Association for the Advancement of Medical Instrumentation (AAMI) and International Organization for Standardization (ISO) in 2018, the tolerable error of blood measurement is equal to or less than 10 millimeters of mercury (mm Hg) with an estimated probability of 85% at least.

The blood pressure measurement method can be separated into two categories, namely, cuff-based method and cuffless method. The cuff-based method is intrusive because one of the arms of the user has to be cuffed for at least 30 seconds to obtain an accurate reading. Therefore, cuff-based method is not suitable for a long-term blood pressure measurement, all day for instance. However, the cuff-based sphygmomanometer can accurately measure the blood pressure of the user. On the other hand, the cuffless sphygmomanometer relies on sensors attached to the user's body, the sensors are used for obtaining sensing data of one of the user's electrocardiography(ECG), photoplethysmography(PPG), and Pulse Transit Time(PTT) data, and then the sensing data is converted into a blood pressure value. Since the volume of the sensor is smaller than that of the cuff, the cuffless sphygmomanometer is less interfering and thus can continuously measure blood pressures in a long period. However, since the result of the cuff-based blood pressure measurement is considered “gold standard”, the cuffless blood pressure measurement is naturally less accurate. In addition, the cuffless sphygmomanometer need to collect a plurality of sensing data of the user in a variety of situations such as walking, sitting, exercising in order to provide a relatively accurate blood pressure measurement. Therefore, the user needs to take an extra effort and time to provide sensing data in different situations.

On the other hand, considering the actual application scenario of the wearable blood pressure measurement device, the ECG signal-based blood pressure measurement may not be suitable in some situation, when users are sleeping for instance, for users do not always have spare hands to measure the blood pressure. In addition, users sometimes hope to rapidly acknowledge a self-value of blood pressure measurement when busy, or acknowledge an accurate self-value of blood pressure measurement when in a free time. However, the wearable blood pressure measurement devices nowadays merely contain a single measurement mode, having a single accuracy accordingly, in blood pressure measurement. Overall, the wearable blood pressure measurement devices nowadays lack of flexibility in practical usage.

### SUMMARY

Accordingly, the disclosure provides a method for dynamically switching blood pressure measurement models, providing alternative blood pressure models for specified users for blood pressure measuring according to different condition. On the premise of preserving the advantage of the cuffless sphygmomanometer that can be worn on the body and can continuously measure the blood pressure, the accuracy of blood pressure measurement is improved, and the daily routine of the specified user is less interfered. Compared to the traditional wearable blood pressure measurement device providing only a single type of measurement mode and unchangeable measurement accuracy, one wearable blood pressure measurement device to which the present disclosure is applied has more flexibility in usage.

According to one or more embodiments of this disclosure, a method for dynamically switching blood pressure measurement model, adapted to a wearable blood pressure measurement device with a biosignal sensing assembly and a processor, is provided. The biosignal sensing assembly comprises two exposed electrodes. The method comprises obtaining a potential difference by said two exposed electrodes, determining whether the potential difference is smaller than a potential threshold by the processor, obtaining a first biosignal of the specified user by the biosignal sensing assembly when the potential difference is smaller than the potential threshold, and calculating a first blood pressure value by the processor according to at least the first biosignal and a first blood pressure model and outputting a first blood pressure value, and obtaining a second biosignal of the specified user by the biosignal sensing assembly when the potential difference is not smaller than the potential threshold, wherein types of the first and second biosignal are different, and calculating a second blood pressure value by the processor according to the second biosignal and a second blood pressure model and outputting a second blood pressure value.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a schematic diagram of a wearable blood pressure measurement device to which the present disclosure is adapted.

FIG. 2 is a flowchart of a method for dynamically switching blood pressure measurement models according to an embodiment of the present disclosure.

FIG. 3 is a partial flowchart of a method for dynamically switching blood pressure measurement models according to another embodiment of the present disclosure.

FIG. 4 is a flowchart for performing a minor adjustment on the first blood pressure model to obtain a first specified blood pressure model.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

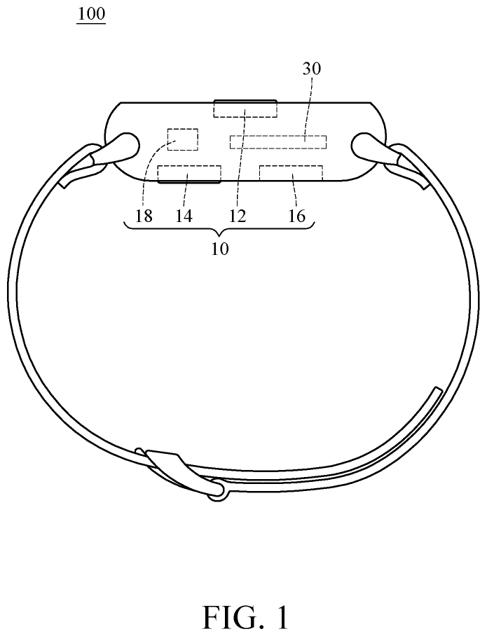

The method for dynamically switching blood pressure measurement models according to an embodiment of the present disclosure is adapted to a wearable blood pressure measurement device preferably. Please refer to FIG. 1, which is a schematic diagram of a wearable blood pressure measurement device 100. The type of the wearable blood pressure measurement device 100 recited in FIG. 1 is cuffless. However, the present disclosure does not limit the structure of the embodiment above.

As recited as FIG. 1, the wearable blood pressure measurement device 100 comprises a biosignal sensing assembly 10 and a processor 30. The biosignal sensing assembly 10 comprises two exposed electrodes 12 and 14, a photoplethysmography(PPG) sensor 16 and a motion sensor 18.

The two exposed electrodes 12 and 14 are disposed at outer surfaces of the wearable blood pressure measurement device 100. The two exposed electrodes 12 and 14 are respectively used for contacting limbs on two sides of the heart of a specified user in order to measure an electrocardiography(ECG) signal. For instance, when said specified user wears the wearable blood pressure measurement device 100, part of the wrist or the back of the hand on one side of the wearable blood pressure measurement device 100 contacts the exposed electrode 14, while the other hand free from the wearable blood pressure measurement device 100 touches the exposed electrode 12 in order to provide an ECG signal.

The PPG sensor is used for measuring a PPG signal.

The motion sensor 18, for example, is a gyroscope or an accelerometer, used for measuring a momentum of itself. In other words, the motion sensor 18 is used for detecting whether the specified user wearing the wearable blood pressure measurement device 100 is moving, thereby determining whether the specified user is in a sleeping status or an active status. In an embodiment, motion sensor 18 is omitted. It is not necessary for the wearable blood pressure measurement device 100 to comprise the motion sensor 18 for performing the method of the present disclosure.

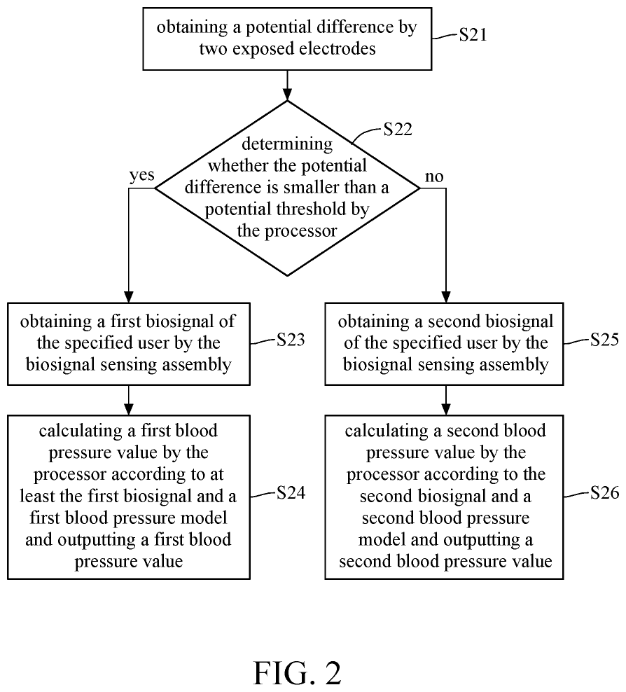

Please refer to FIG. 2, which shows the flowchart of a method for dynamically switching blood pressure measurement models according to an embodiment of the present disclosure.

Please refer to a step S21, obtaining a potential difference by the two exposed electrodes 12 and 14. Step S21 is used for determining whether the specified user performs an ECG based blood pressure measurement actively or not.

Please refer to a step S22, determining whether the potential difference is smaller than a potential threshold by the processor 30. When the specified user contacting the two exposed electrodes 12 and 14 respectively with limbs on two sides of the heart of the specified user, said two exposed electrodes 12 and 14 and said specified user form a path jointly, thus having a potential difference formed between the exposed electrodes 12 and 14. The processor 30 determines whether the potential difference is smaller than a predetermined potential threshold. If the determination result is positive, please go to a step S23; otherwise please go to a step S25. Practically, when the specified user does not contact the two exposed electrodes 12 and 14 respectively with limbs on two sides of the heart of the specified user, said two exposed electrodes 12 and 14 and said specified user don't form the path, so the processor 30 is not able to detect any potential difference. In other words, the value of the potential difference which the processor 30 detects is infinity.

The positive determination result of the step S22 as that “the potential difference is smaller than the threshold” means the processor 30 confirms that the specified user wants to perform an ECG based blood pressure measurement. Please refer to a step S23, obtaining a first biosignal of the specified user by the two exposed electrodes 12 and 14 of the biosignal sensing assembly. In an embodiment, the first biosignal is an ECG signal. In another embodiment, the first biosignal is a synchronous signal formed with the ECG signal and the PPG signal. In the step S23 of this another embodiment, obtaining PPG signal by the PPG sensor is needed besides obtaining the ECG signal by the two exposed electrodes 12 and 14.

Please refer to a step S24, calculating a first blood pressure value by the processor 30 according to at least the first biosignal and a first blood pressure model. Said first blood pressure model is a general blood pressure model restored in the processor 30 in advance. More specifically, before obtaining said potential difference by said two exposed electrodes 12 and 14, a plurality of first physiological data, a plurality of second physiological data and a plurality of first blood pressure data of a plurality of general users are obtained in advance. The first physiological data, for instance, are ECG signals obtained from the general users. The second physiological data, for instance, are PPG signals obtained from the general users. The first blood pressure data, for instance, are blood pressure values of the general users measured by traditional sphygmomanometer. Said blood pressure values include systolic blood pressure values and diastolic blood pressure values.

In an embodiment, building the first blood pressure model according to said first physiological data and said first blood pressure data by performing a deep learning algorithm, and building the second blood pressure model according to said second physiological data and said first blood pressure data by performing the deep learning algorithm are performed. Said deep learning algorithm is a convolutional neural network which adopts multilayer perceptron as a regressor. Said first blood pressure model is based on the training of the ECG signals and said first blood pressure data are from the general users. Said second blood pressure model is based on the training of the PPG signals and said first blood pressure data are from the general users.

In another embodiment, said first blood pressure model is the result of a training based on the ECG signals, the PPG signal and said first blood pressure data from the general users. Said second blood pressure model is the result of a training based on the PPG signals and said first blood pressure data from the general users.

In a further embodiment, calculating Pulse Transit Time(PTT) based on the ECG signals and the PPG signals from the general users in advance is performed, and said first blood pressure model is the result of a training based on both the PTT signals and said first blood pressure data from the general users.

Please refer to a step S25, when the result of the step S22 is that “the potential difference is not smaller than the threshold”, obtaining a second biosignal, for instance, a PPG signal, of the specified user by the PPG sensor 16 of the biosignal sensing assembly 10. The type of the second biosignal differs from that of the first biosignal.

Please refer to a step S26, calculating a second blood pressure value according to the second biosignal and a second blood pressure model by the processor 30.

In the embodiment above, the wearable blood pressure measurement device 100, for instance, determines whether the specified user wants to perform an ECG-signal-based blood pressure measurement. For example, when the specified user contacts the two exposed electrodes 12 and 14 with two hands, the processor 30 may select the first blood pressure model and calculate a first blood pressure value according to at least the first biosignal. Said first blood pressure model, for instance, is the result of a training based on the ECG signals and the PPG signals from the general users, or based on the PTT signals, therefore having higher accuracy of the measurement. However, the processor 30 may select a first blood pressure model based only on the ECG signals. For another example, when the specified user is unable to contact both two exposed electrodes 12 and 14 at the same time while sleeping, the processor 30 may select the second blood pressure model and calculate a second blood pressure value according to the second biosignal. the measurement according to the PPG signal can be performed when the specified user cannot provide the ECG signal, for it does not interfere the daily routine of the specified user. Overall, a method for dynamically switching blood pressure measurement according to the embodiment above of the disclosure dynamically provides the method of blood pressure measurement with higher accuracy or less interference, therefore it can be adapted to alternative usage scenario.

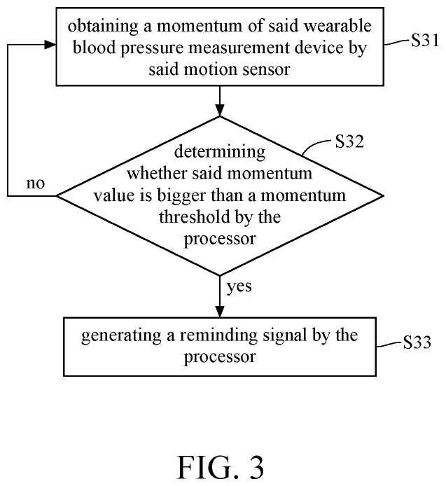

Please refer to FIG. 3, which shows a partial flowchart of a method for dynamically switching blood pressure measurement model according to another embodiment of the present disclosure. The process described below is selectively applied before the step S21 recited in FIG. 2. However, the present disclosure is not thus limited.

Please refer to a step S31, obtaining a momentum of wearable blood pressure measurement device 100 by the motion sensor 18. More specifically, detecting the movement of the specified user before obtaining the potential difference by the two exposed electrodes 12 and 14 is performed in advance.

Please refer to a step S32, determining whether said momentum value is bigger than a momentum threshold by the processor 30. In an embodiment, in addition to determining the motion status detected by the motion sensor 18 by the processor 30, the step S32 further comprises the step of determining the continuous time period in which the momentum is higher than the momentum threshold by the processor 30.

Please refer to a step S33, when the momentum is higher than the momentum threshold, generating a reminding signal by the processor 30 for reminding that the specified user contacts said two exposed electrodes with two body parts. After the step S33, the processor 30 performs the step S21 recited in FIG. 2 in order to obtain the potential difference. In contrary, when the momentum is not higher than the momentum threshold, please go back to the step S32, continuously determining the momentum detected by motion sensor 18 by the processor 30.

Practically, when the specified user is not under sleeping status, the wearable blood pressure measurement device 100, in the step S32, may detect slight movement of the specified user. Under the premise that the specified user is not under sleeping status, the wearable blood pressure measurement device 100 may generate a reminding signal in order to ask the specified user whether to adopt the blood pressure measurement model having higher accuracy, for example, the blood pressure measurement model based on the trained ECG signals and PPG signals, or the blood pressure measurement model based on the trained PTT signals, or the blood pressure measurement model merely based on the trained ECG signals. The reminding signal may as well be used for users for selecting either of the above models to perform the blood pressure measurement. In another embodiment, under the premise of determining that the specified user is not under sleeping status, the wearable blood pressure measurement device 100 may automatically switch to the blood pressure measurement model based on the trained both ECG signals and PPG signals or to the blood pressure measurement model based on the trained PTT signals in order to perform the following blood pressure measurement. In this another embodiment, said method may reduce the interference of the wearable blood pressure measurement device 100 to the specified user, and preserve the flexibility of dynamically switching blood pressure measurement model.

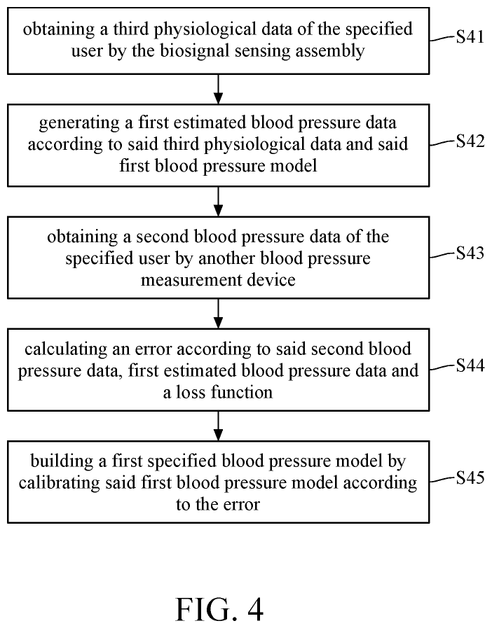

Please refer to FIG. 4, which shows the flowchart for performing the minor adjustment of the first blood pressure model to obtain the first specified blood pressure model. The measurement accuracy of the first blood pressure model may further increase through the process recited in FIG. 4.

In an embodiment, besides building general blood pressure model according to physiological data of a plurality of general user, said method further calibrates the general blood pressure model according to the specified user, making it more adapted to the physiological status of the specified user and builds a customized blood pressure model. Previously described first blood pressure model taken as an example, the following describes the steps of calibrating the first specified blood pressure using the first blood pressure model. Person having ordinary skill in the art may adaptively calibrate the second specified blood pressure using the second blood pressure model by modifying the steps recited in FIG. 4.

Please refer to a step S41, obtaining a third physiological data of the specified user by the biosignal sensing assembly 10 of the wearable blood pressure measurement device 100. Said third physiological data, for instance, is a ECG signal, a PPG signal, a time synchronous signal of both ECG signal and PPG signal, or a PTT signal. The type of the third physiological data is same as that of the training first blood pressure model.

Please refer to a step S42, generating a first estimated blood pressure data according to said third physiological data and said first blood pressure model. In an embodiment, the first blood pressure model comprises a parameter set and a loss function. When adopting the neural network to build the first blood pressure model, a set of weights are served as said parameter set. When adopting the linear regression to build the first blood pressure model, a set of every parameter of linear function are served as said parameter set. In the step S42, the output after substituting the third physiological data into the parameter set of the first blood pressure model is served as a first estimated blood pressure data. Said output, for instance, is a systolic blood pressure(SBP) or a diastolic blood pressure(DBP), depending on whether the first blood pressure data previously trained is a SBP or a DBP.

Please refer to a step S43, obtaining a second blood pressure data of the specified user by another blood pressure measurement device. Said another blood pressure measurement device, for instance, is a sphygmomanometer.

Please refer to a step S44, calculating an error according to said second blood pressure data, first estimated blood pressure data and the loss function.

In an embodiment, method for calculating the error is as the Formula. 1 below:

L_{general}=∥BP− [symbol] ∥₂² (Formula. 1)

The L_{general} is the loss function of the first blood pressure model, the BP is the second blood pressure data of the specified user and is either a value of the SBP or a value of the DBP measured by another blood pressure measurement device, and the BP is the first estimated blood pressure data. The process aims to train a useable first blood pressure model by minimizing the loss function.

Please refer to a step S45, calibrating said first blood pressure model according to the error in order to build a first specified blood pressure model. If the first blood pressure model, for instance, is a linear model, the data points drawn according to the third physiological data and the second blood pressure data are not necessarily perfectly on the curve corresponding to said linear model. Therefore, the step S45 describes how to adaptively modify the curve of the linear model, so as to minimize the error between said curve and the data points of the specified user and then obtain the first specified blood pressure model. In order to obtain the first specified blood pressure model through the learning method, a process of regularization can be performed, as the Formula. 2 in below:

L_{calibration}=L_{general}+λ_{reg}L_{reg} (Formula. 2)

The L_{calibration} is the loss function of the first specified blood pressure model which is estimated obtained after calibrated. The λ_{reg} is an adjustable parameter. The bigger the λ_{reg} is set, the bigger the similarity between the first specified blood pressure model and the first blood pressure model is. If the λ_{reg} is set to 0, the curve corresponding to the first blood pressure model and the data points of the specified user would fully coincide with each other. The L_{reg} is the modification function of the regularization process, whose calculating method is shown in the Formula. 3 as below. In order to maintain the characteristics of the first blood pressure model, preventing the loss function from being dominated by the data points of the specified user, thus the L_{calibration} is being calibrated through the L_{reg} and the pre-determined λ_{reg}.

L_{reg}=∥θ_{general}−θ_{subject}∥₁¹ (Formula. 3)

Wherein the θ_{general} is a set of weights of the first blood pressure model, the θ_{subject} is a set of weights of the first specified blood pressure model. To ensure that the θ_{subject} does not deviate from the originally learned the θ_{general}, an embodiment of the present disclosure employs an L1-regularization in order to preserve the weight of biggest contribution to the first estimated blood pressure data.

According to the error obtained in the step S44, and selecting appropriate adjustable parameter the λ_{reg}, said method can optimize the loss function of the first specified blood pressure model, then building the first specified blood pressure model adapted to the specified user.

In view of the above description, the disclosure provides a method for dynamically switching blood pressure measurement model, automatically switching suitable (or selected by the specified user) blood pressure models for blood pressure measuring according to different condition. On the premise of preserving the advantage of the cuffless-based sphygmomanometer that can be worn on the body and can continuously measure, through the steps of calibration according to both the physiological data and the blood pressure data of the specified user, the accuracy of the blood pressure measurement model for blood pressure measuring is improved, and the specified user is less interfered. Compared to the traditional wearable blood pressure measurement device providing only a single type of measurement mode and fixed measurement accuracy, the wearable blood pressure measurement device to which the present disclosure is applied has more flexibility in usage.

What is claimed is:

1. A method for dynamically switching blood pressure measurement models, adapted to a wearable blood pressure measurement device with a biosignal sensing assembly and a processor, wherein said biosignal sensing assembly comprises two exposed electrodes, with said method comprising:
obtaining a potential difference by said two exposed electrodes;
determining whether the potential difference is smaller than a potential threshold by the processor;
obtaining a first biosignal of the specified user by the biosignal sensing assembly when the potential difference is smaller than the potential threshold;
calculating a first blood pressure value by the processor according to at least the first biosignal and a first blood pressure model and outputting a first blood pressure value;
obtaining a second biosignal of the specified user by the biosignal sensing assembly when the potential difference is not smaller than the potential threshold, wherein types of the first and second biosignal are different; and
calculating a second blood pressure value by the processor according to the second biosignal and a second blood pressure model and outputting a second blood pressure value.

2. The method for dynamically switching blood pressure measurement models as recited in claim 1, wherein when determined that the potential difference is smaller than the potential threshold by said processor, said method further comprises:
obtaining said second biosignal by said biosignal sensing assembly; and
wherein calculating said first blood pressure by the processor according to at least the first biosignal and a first blood pressure model comprises:
calculating said first blood pressure by the processor according to said first biosignal, said second biosignal and said first blood pressure model.

3. The method for dynamically switching blood pressure measurement models as recited in claim 1, wherein
said first biosignal is an electrocardiography signal; and
said second biosignal is a photoplethysmography signal.

4. The method for dynamically switching blood pressure measurement models as recited in claim 2, wherein
said first biosignal is an electrocardiography signal; and
said second biosignal is a photoplethysmography signal.

5. The method for dynamically switching blood pressure measurement models as recited in claim 1, wherein said wearable blood pressure measurement device further comprises a motion sensor, and before obtaining said potential difference by said two exposed electrodes, said method further comprises:
obtaining a momentum of said wearable blood pressure measurement device by said motion sensor;
determining whether said momentum value is bigger than a momentum threshold by the processor; and
generating a reminding signal for reminding that the specified user contacts said two exposed electrodes, when said momentum value is bigger than the momentum threshold.

6. The method for dynamically switching blood pressure measurement models as recited in claim 1, wherein before obtaining the potential difference by said two exposed electrodes, said method further comprises:
obtaining a plurality of first physiological data, a plurality of second physiological data and a plurality of first blood pressure data from a plurality of general users;
building the first blood pressure model according to said first physiological data and said first blood pressure data by performing a deep learning algorithm; and
building the second blood pressure model according to said second physiological data and said first blood pressure data by performing the deep learning algorithm.

7. The method for dynamically switching blood pressure measurement models as recited in claim 1, wherein before obtaining the potential difference by said two exposed electrodes, said method further comprises:
obtaining a plurality of first physiological data, a plurality of second physiological data and a plurality of first blood pressure data from a plurality of general users;
building the first blood pressure model according to said first physiological data by performing a deep learning algorithm, said second physiological data and said first blood pressure data; and
building the first blood pressure model according to said second physiological data and said first blood pressure data by performing a deep learning algorithm.

8. The method for dynamically switching blood pressure measurement models as recited in claim 6, wherein after building the first blood pressure model, said method further comprises:
obtaining a third physiological data of the specified user by said biosignal sensing assembly;
generating a first estimated blood pressure data according to said third physiological data and said first blood pressure model;
obtaining a second blood pressure data of the specified user by another blood pressure measurement device;
calculating an error according to said second blood pressure data, first estimated blood pressure data and a loss function; and
building a first specified blood pressure model by calibrating said first blood pressure model with the error.

9. The method for dynamically switching blood pressure measurement models as recited in claim 6, wherein after building the second blood pressure model, said method further comprises:
obtaining a third physiological data of the specified user by said biosignal sensing assembly;
generating a first estimated blood pressure data according to said third physiological data and said second blood pressure model;
obtaining a second blood pressure data of the specified user by another blood pressure measurement device;
calculating an error according to said second blood pressure data, first estimated blood pressure data and a loss function; and
building a second specified blood pressure model by calibrating said first blood pressure model with the error.

10. The method for dynamically switching blood pressure measurement models as recited in claim 6, wherein said deep learning algorithm is a convolutional neural network which adopts multilayer perceptron as a regressor.
